(19) United States
(12) Patent Application Publication
(10) Pub. No.: US 2021/0038153 A1
(43) Pub. Date: Feb. 11, 2021

(54) Removable Orally Insertable Device with Usage and Location Tracking

(71) Applicants: Lynne Claflin, Kendalia, TX (US); Jon Cameron, Dallas, TX (US)

(72) Inventors: Lynne Claflin, Kendalia, TX (US); Jon Cameron, Dallas, TX (US)

(73) Assignee: Lynne Claflin, Kendalia, TX (US)

(21) Appl. No.: 17/069,925

(22) Filed: Oct. 14, 2020

(51) Int. Cl. A61B 5/00

(52) U.S. Cl. CPC A61B 5/682; A61B 2560/0214; A61B 5/0004; A61B 5/0022

(57) ABSTRACT

A system and method to track the location and usage time of an orally insertable orthodontic device. The switch attached to the orthodontic device reports to the user device when it was inserted and removed from the mouth. Once removed from the mouth, the orthodontic device transmits a signal used to geolocate the device in the event it has been lost by the patient.

### BACKGROUND

### Field of the Invention

The invention related to the system and method of tracking and recording the usage of an orally insertable orthodontic device and tracking the device's position within a geographic space.

### Background of the Invention

The present invention is a useful and novel method and system for informing parties when the orthodontic device is inserted in the patient's mouth and removed from the patient's mouth. When the device is removed from the mouth, a switch in the orthodontic device allows a radio to issue a communications signal suitable for tracking the location of the device. The communication is made through a connection to a master central device, or user device, such as a smartphone. An application on the smartphone provides both a diary and location mapping controls. Mapping controls may monitor the location of the orthodontic device, track past locations the orthodontic device, predict the location of the orthodontic device, provide an alert when the orthodontic device and mobile phone are separated.

The present invention solves three common pain points associated with the use of orally insertable orthodontic device:
1. Tracking the total usage time by the patient;
2. Reminding the patent to reinsert the orthodontic device;
3. Locating an orthodontic device, such as a retainer, when it has been misplaced by the patient. This is an increasing problem as retainers are becoming clearer in color, by design, and therefore difficult to visually locate.

### SUMMARY OF THE INVENTION

An invention, which meets the needs stated above, is a system and method to monitor the usage and location of orally insertable orthodontic device. In order to preserve battery power, the electronic device encased in the orthodontic device contains a switch that reports if the device has been inserted and removed from the mouth. Once removed from the mouth, the orthodontic device transmits a signal used to geolocate the device in the event it has been lost by the patient.

### Objects and Advantages

Accordingly, besides the objects and advantages of the system and methods for a switched orally insertable orthodontic device, as described above, several objects and advantages of the present invention are:
a) to provide orthodontists with a new tool to monitor patient's compliance with usage instructions;
b) to provide patients with a simplified application that automatically tracks their usage of an orally insertable orthodontic device;
c) to provide patients with real-time reminders that they have not re-inserted the device.

Further objects and advantages of this invention will become apparent from a consideration of the drawings and the ensuing description of the drawings.

### DRAWING FIGURES

The accompanying drawings, which are incorporated in and constitute a part of this specification, illustrate embodiments of the present invention and together with the description, serve to explain the principles of this invention. In the figures:

FIGS. 1A-1C—Drawings of example master central device functions.

FIG. 2—Illustration of the components of the orally insertable orthodontic device and master central device.

FIG. 3A-3B—Figures showing the electronic components of an example orally insertable orthodontic device.

FIG. 4A-4B—Graphics showing how the device locates the orally insertable orthodontic device.

### KEY TERMS

Bluetooth Low Energy (BLE): wireless personal area network technology designed and marketed by the Bluetooth Special Interest Group (Bluetooth SIG) aimed at applications in the personal communications, healthcare, beacon, fitness, security, and home entertainment industries. Bluetooth Low Energy provides considerably reduced power consumption and cost while maintaining a similar communication range.

BLE SoC: Bluetooth Low Energy System on Chip. A chipset containing layers of functions such as radio, application, controller and processor.

False North/South axis: A line created by the user device's application that is unassociated with true north.

Master central device: an electronic device used to collect, store, interpret and display data transmitted from the orally insertable orthodontic device by the device's electronics.

Orally insertable orthodontic device: any device intended with a use related to teeth. This can include retainer, bridge, dentures, and braces.

### REFERENCE NUMERALS IN DRAWINGS

10 Device components
50 Mouth
60 Saliva
70 Tooth
100 Orally insertable orthodontic device, retainer, bridge, dentures, braces
120 Power generator, titanium rod
130 Battery
140 Connector, rectifier, inverter, converter
150 Switch, deforming switch
160 Deforming contacts
170 Switch data
200 System on Chip (SoC), BLE System on Chip (BLE SoC), Bluetooth System on Chip, chipset, communication chip
210 Processor
220 Radio, BLE radio
230 Memory
240 Peripherals
250 Identification
290 Connection
300 Master central device, user device
310 Bluetooth, communication chipset
320 Application
330 Storage
335 Database
340 Processor
350 Memory
360 Setup controls, setup
370 Mapping application
380 Power
390 SoC selection controls
395 Location services module
400 Patient, user
500 Advertising devices
505 Advertising data
510 Received signal strength (RSSI) in Decibel milliwatts (dBm)
520 Alphanumeric string name
530 Device address (Bluetooth)
540 Connection interval
600 Geographic space
610 Adverting device ‘DMM 72134’
620 Advertising device ‘Lisa's iPhone’
630 Advertising device ‘LiftMaster’
650 False North/South axis

### DETAILED DESCRIPTION OF THE DRAWINGS

Referring to the drawings, in which like numerals represent like elements,

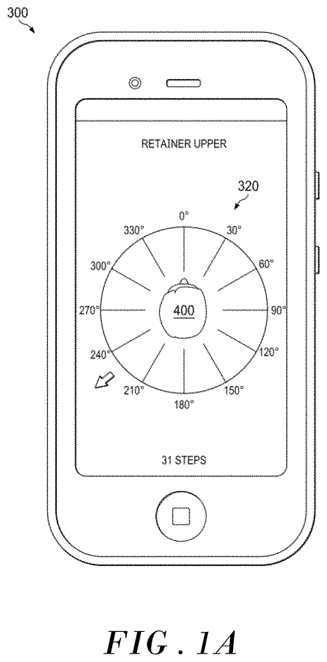

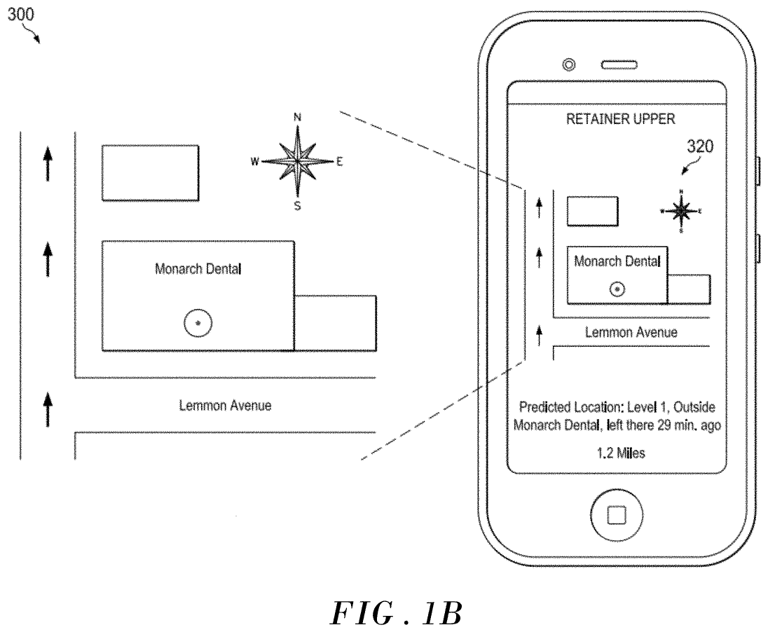

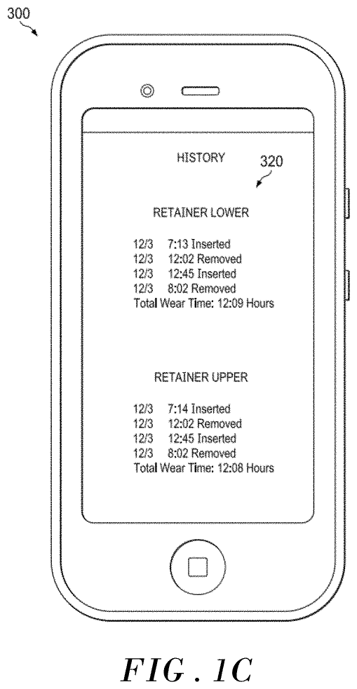

### FIGS. 1A-1C

FIGS. 1A to 1C depict the master central devices 300 example functions of the present invention.

First turning to FIG. 1A showing a non-limiting example application 320 on the user device 300 for utilization by the user 400 to manage the functions of an orally insertable orthodontic device 100. Orally insertable orthodontic devices 100 may comprise a retainer, bridge, dentures, and braces. The setup controls 360 comprise functions to allow the adding the IP address of orally insertable orthodontic devices 100, delete devices 100, name the devices 100 and location services module 395. In a non-limiting example application 320, the user 400 would select setup controls 360 to add a device 100. To match the retainer 100, the user 400 launches the setup 360 and locates the device 100 within the list of advertising devices 500. The identification 250 which may comprise advertising data 505 including the full identification serial number, machine ID, universal unique ID (UUID) physical address, MAC address, IP address, device's alphanumeric local name, access address, service, GATT profile, general attribute profile, Bluetooth device address 530, or the raw advertising data 505. The device application 320 would store the association and the description on the user device 300 in the database 335 in storage 330. The description may be assigned by the patient 400, dental professional or manufacturer. Exiting the setup screen 360, the user 400 now shows ‘connected’ to one or more orthodontic devices 100. If more than one, the application 320 shows the advertising data 505 from a System on Chip (SoC) 200 to allow the user 400 to select the specific orthodontic device 100. The System on Chip is variously referred to in the industry as an SoC, BLE System on Chip (BLE SoC), Bluetooth System on Chip, chipset, communication chip.

In an orthodontic device 100 to master central device 300 configuration, the user 400 would first turn on the master central device 300, launch the application 320 which then displays historical data and/or provides tracking services to locate one or more orthodontic device 100. To locate the orthodontic device 100, the application 320 would establish a new connection 290 to the BLE radio 220 using the master central device's 300 Bluetooth connection 310. The master central device's 300 Bluetooth 310 connects to the orthodontic device's BLE radio 220 and establishes a wireless communication. The master central device 300 may then send a pairing code to the BLE radio 220 and in turn receives a pairing confirmation to complete the pairing process. In a preferred embodiment, the BLE radio 220 and master central device 300 are bonded in the previous setup 360.

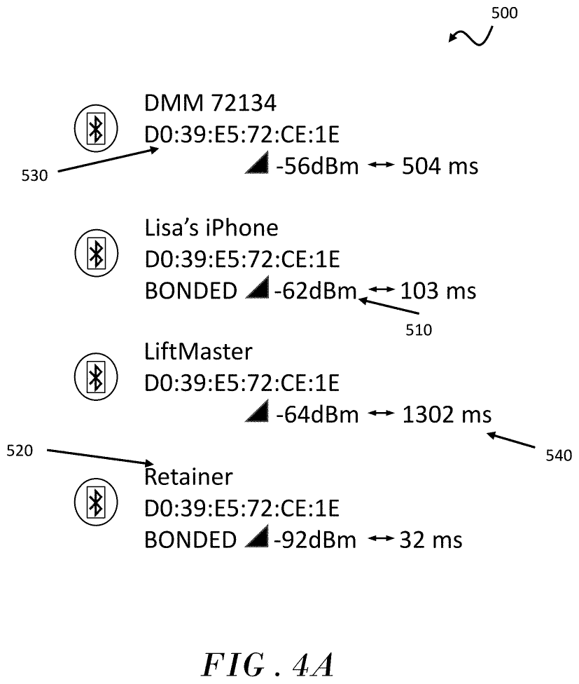

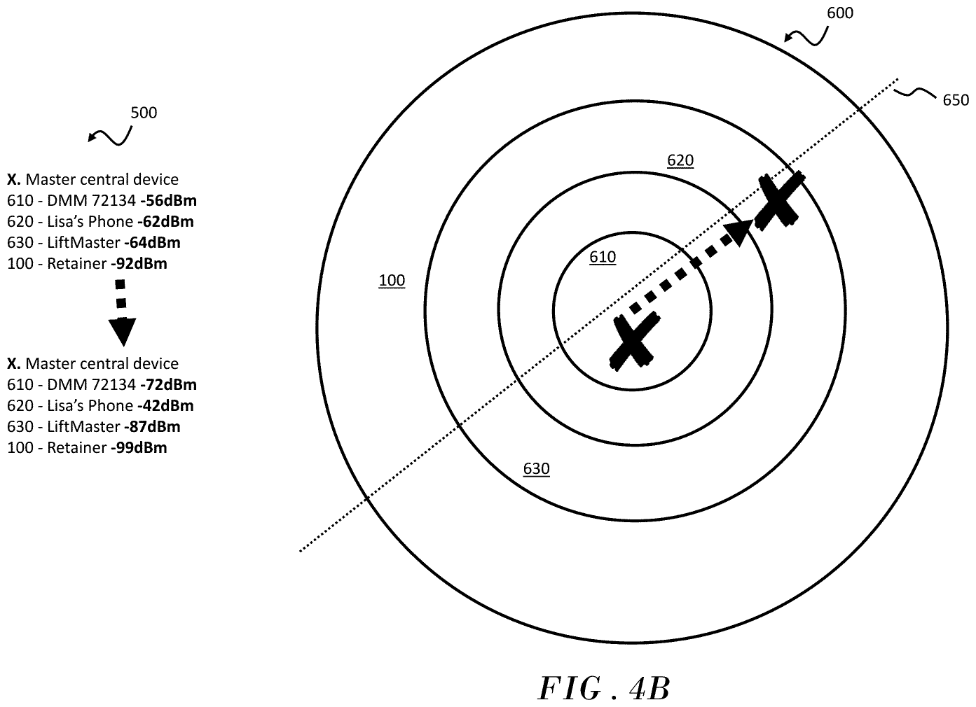

The user 400 selects the user-assigned description of ‘RETAINER UPPER’ using the Bluetooth System on Chip (BLE SoC) 200 selection controls 390 on the user device 300. The patient 400 can now engage the location services module 395 on the application 320. The location services module 395 performs the functions of providing location information to the application 320 including coordinating with the user device's 300 GPS and Bluetooth services. In the present non-limiting example, the application 320 shows a simple graphic with the user 400 graphically represented in the center of the compass. The user begins moving around a space 600 as the device stores signal strengths of other advertising devices 500 in the room. Using a triangulation method, the display directs the user 400 to the direction, and ideally the distance, to the orthodontic device 100. The user 400 moves through the geographic space 600 to provide additional data points to the application 320. Once the orthodontic device 100 is located, the user 400 can close the application 320 or chose a new orthodontic device 100 with the BLE SoC 200 selection controls 390. See FIGS. 4A-4B for further discussion of the location services module 395.

In another preferred method in FIG. 1B, the application 320 shows a map of the orthodontic device's 100 location based on the last paired data of the orthodontic device's 100 static location and the master central device's 300 GPS recorded location, using the location services module 395 of the application 320, at the time of the recording of the connection 290 to the orthodontic device 100. In the event the orthodontic device 100 is not transmitting from the BLE SoC 200, or is not within effective distance, the user device 300 would retrieve the Decibel milliwatts (dBm) 510 of the last communication 290 from the storage 330, recorded from the BLE SoC 200 and pair it with the GPS location of the master central device 300 at the time of the last communication 290. Global Positioning System, GPS, is a technology that informs the user device's 300 location and provides information about a given point on earth. GPS consists of three segments: the space segment, the ground segment and receivers. The space segment consists of a network of more than thirty satellites that constantly orbit the earth and emit microwave signals that are captured by the antenna installed inside the user device 300 or any other GPS-enabled device 300. Using only four satellites, trilateration works to estimate a GPS-enabled device's exact location with accuracy that can be plus or minus one meter. Trilateration is a mathematical technique used by a global positioning system (GPS) device to determine user position, speed, and elevation. Another or additional method for position calculation is triangulation. Triangulation not only calculates the distances to the sensor but the angles to the sensor to determine the position of an object.

The application 320 may also use the paired data of GPS and RSSI 510 to display the last known location on the location services module 395 which may include descriptive data.

In FIG. 1C, the application 320 of the master central device 300 may use data provided by a switch 150 in the orthodontic device 100 where the switch 150 provides data of when the device is inserted and removed from the mouth 50. The history of the switch's 150 on and offs can be recorded by the master central device 300 during periodic communication connections 290 with the orthodontic device 100. The communications between the two devices 100, 300 may be timed as scheduled, continuous, or intermittent such as in a slave/master communication. The switch 150 provides data on when the patient 400 has inserted the orthodontic device 100 and subsequently removed the orthodontic device 100 from the mouth 50.

By recording the insertion time and removal time in the storage 330 or memory 350 of the master central device 300, a diary of the total usage time by the patient 400 may be developed to improve and monitor treatment. In another embodiment, the monitoring of the patient's 400 scheduled use in real time allows the master central device 300 to provide an alert that the orthodontic device 100 needs to be re-inserted in the mouth 50 after events such as mealtimes and waking.

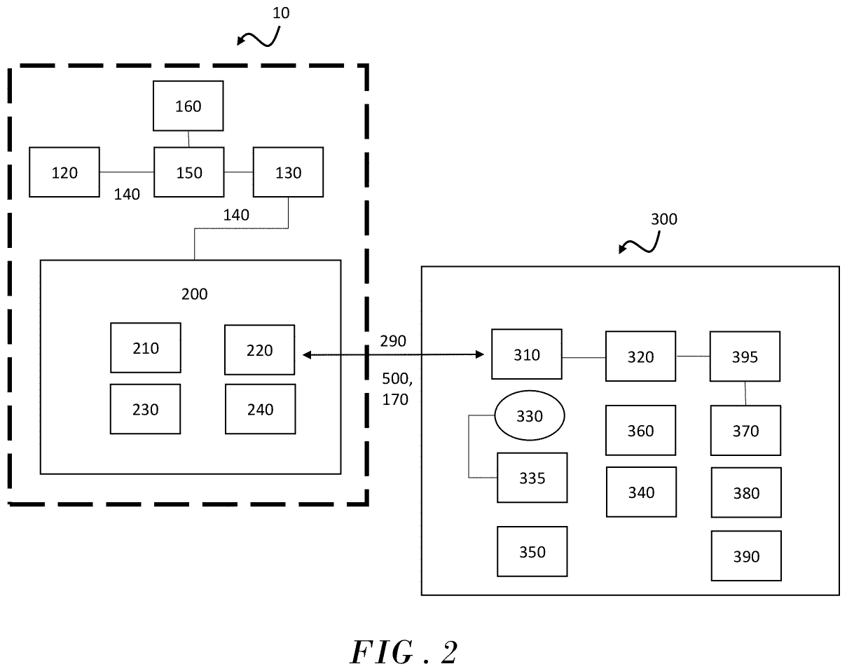

### FIG. 2

FIG. 2 is a systems overview of the orthodontic device 100 and master central device 300. The orthodontic device components 10 include a power generator 120 with a connector 140 to a battery 130 to store the power generated by the power generator 120. Orthodontic devices 100 using any type of metal alloy contain all the necessary ingredients to create an environment to charge a battery 130 in the mouth 50 when combined with saliva 60. Titanium 120 is commonly used for dental repairs that can create what is called ‘oral galvanism’ or ‘the battery effect’ in the mouth 50. The process of oral galvanism with titanium implants releases metal ions into the mouth 50 and jawbone constantly when it comes into contact with saliva 60. Other types of preferred power generators 120 for the orthodontic dental devices 100 include nanogenerators (mechanical or thermal properties to create small-scale energy production), piezoelectric materials (applied mechanical stress), such as jaw activity or opposing jaw pressure. The battery 130 is miniaturized and be an appropriate implantable medical device such as the Sandia National Laboratory Nano Battery.

The power generator 120 uses one or more connectors 140 to attach to the battery 130. In a preferred embodiment, when a chipset 200 senses wireless radiation, it may wake up and harvests those signals and turns them into a new power source. Connectors 140 may be wired or wireless. Connectors 140 may comprise wires, rectifier, converter, and inverter. A switch 150 may be located between the battery 130 and power generator 120 so the battery 130 can power the BLE SoC 200 when the orthodontic device 100 is not generating energy. The battery 130 may be located as a layer on the Bluetooth SoC 200.

The battery 130 may also have a connector 140 to a switch 150 with an additional connector 140 to a communication chip 200, such as a BLE SoC 200. The chipset 200 comprises layers such a processor 210, battery 130, radio 220 (such as a BLE radio), memory 230, and peripherals 240. Peripherals 240 comprise voice controls, speakers, lights, General Purpose Input Output (GPIO), pulse width modulation (PWM) control circuit, Universal Asynchronous Receiver/Transmitter (UART), Serial Peripheral Interface (SPI), and inter-integrated circuits (12C).

The hardware of the user device 300 comprises a display for input and output, memory 350 and/or storage 330 (such as ROM and/or RAM), processor 340, a power source 380, communications chipset 310, such as Bluetooth 310, and a module for the device application 320. The power 380 comprises alternating current (AC) and variable direct current (DC) electronically linked together. The display serves the function of rendering the application 320 on the hardware 300 and receiving commands comprising keyboard, chipset 310 selection, setup 360, and communication with the orally insertable orthodontic device 100. The processor 340 runs the application 320 in memory 350 comprising commands such as location services and a use diary in the application 320. The processor 340 also manages the device's 300 communication chipset's 310 communication with the orthodontic device's chipset 200. The processor 340 would manage any mapping applications 370 native to the device 300 or as a module of the device application 320. A database 335 located on the storage 330 stores the setup 360 information, including any mapping application 370 data. The database 335 would record the written description and the association with the orthodontics device's 100 BLE SoC's 200 identification 250.

A master central device 300 comprises any system with a computer processor 340 including mobile computers, personal computers, personal digital assistants, smart phones, laptops, tablets, wearable computers, ultra-mobile personal computers, enterprise digital assistants, electronic book readers, minicomputers, mainframes, servers, workstations, minicomputers, microcomputers, desktop computers, clones, terminals, and the like.

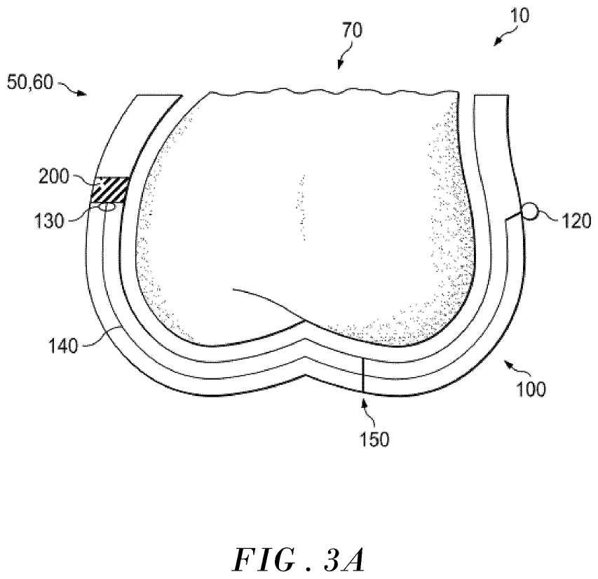

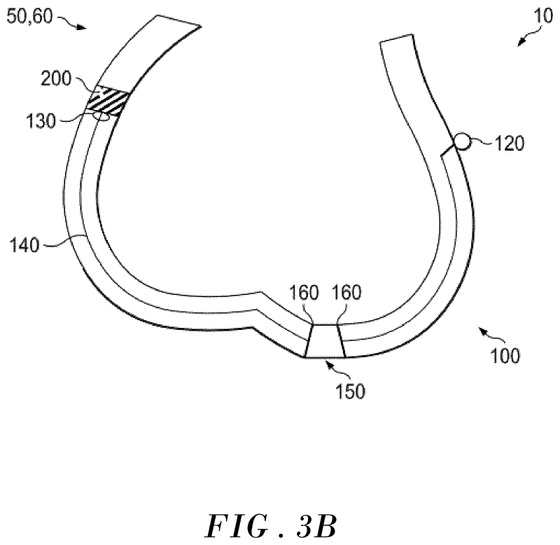

### FIGS. 3A-3B

FIGS. 3A to 3B illustrate the design of a switch 150 and the electronic components associated with the orthodontic device 100. The illustrated switch 150 is a deforming and reforming switch 150. As shown in 3A, the orthodontic device 10 is placed in the mouth 50 with an adjacency to the patient's 400 teeth 70. The orthodontic device 100 comprises various combinations of BLE SoC 200; deforming switch 150 with deforming contacts 160; connectors 140 such as wires, rectifier, inverter, converter; a power generator 120 such a titanium rod 120; and a battery 130. In FIG. 3A, the orthodontic device 100 is inserted over the tooth 70 which presses the sides of the device 100 apart and connects the two deforming contacts 160. This creates a connection a to a BLE SoC 200 and begins the process of charging the battery 130. The BLE SoC 200 collects the relationship between the deforming contacts and stores the switch data 170 in memory 230 to be transmitted to the master central device 300.

The BLE SoC 200 may also inform the BLE radio 220 to cease transmission and records the switch data 170 on the memory 230. In a preferred embodiment, when the power sources, 120, 130 are connected to the chipset 200, the chipset 200 first establishes a connection 290 with master central device 300 and informs of an impending shutdown and allows the master central device 300 to record the switch data 170.

In another preferred embodiment the BLE SoC 200 uses the power from the battery 130 to maintain a connection 290 to the master central device 300 to continually, or intermittently, transmit advertising data 505 and the switch data 170; the switch data 170 which comprises changes in the connection between the deforming contacts 160.

In FIG. 3B, the orthodontic device 100 is removed from the mouth 50 and the device 100 deforms and separates the deforming contacts 160. This generates a connection 290 to the user device 300 and reports the orthodontic device 100 has been removed from the mouth 50. Once removed, the battery 130 is responsible for delivering power to the chipset 200 for the connections 290 to the master central device 300.

Switches 150 may comprise both mechanical switches and electronic switches. For example, the BLE SoC 200 may be turned off by an oral galvanism when a titanium rod 120 comes in contact with saliva 60 and begins to generate energy. The processor 210 on the BLE System on Chip 200 may turn off a BLE radio 220 when the system 10 is recharging the battery 130. The timing of the powering down the BLE radio 220 may be recorded in memory 230 for transmission to the master central device 300 later. In another preferred embodiment the radio 220 maintains a periodic connection with the master central device 300.

Other types of effective switches 150 comprise pressure switches, temperature, heartbeat detector, and light switches.

### FIGS. 4A-4B

Finally, FIGS. 4A-4B are the representation of the data and methods for tracking of an orally insertable orthodontic device 100 using a Bluetooth standard. In FIG. 4A, four representative advertising data set 505 are shown including the listed names of ‘DMM 72134”, “Lisa's iPhone”, “LiftMaster”, and “Retainer”. These representative transmissions are within distance of the master central device 300 and are advertising 505 their presence.

Advertising packets can vary by design and changing standards over time. In this example, the dataset includes device address 530 (in this example, Bluetooth device address 530, connection interval 540 in milliseconds (ms) and the Received Signal Strength (RSSI) shown in Decibel milliwatts 510. The location services module 395 collects advertising data 505 and GPS data and provides the calculations described below to locate the orally insertable orthodontic device 100.

Received signal strength (RSSI) in Decibel milliwatts (dBm) 510 is the relative strength of the signal being transmitted by the retainer 100. The definition of RSSI is ‘total received wide-band power by UE.’ A unit of measure used to reference signal strength to electrical power level. Unlike dB, which is a relative measure, dBm is an absolute measure, and can be used to express very small values (dBm) and very large values (dBW). The baseline reference relationship is 1 mW=0 dBm. It is a ‘distance value.’ The lower the value, the closer orthodontic device 100 is to the user 400. It provides a roughly circular set of possibilities. RSSI 510 is affected by many factors like obstacles, multipath fading, antenna polarization and cross-body shielding. The theoretical relationship between RSSI 510 and distance is:

RSSI [dBm]=−(10.η.log(d)+A) where
η=path loss exponent
d=the distance between node
A=received signal strength in dBm at one meter

The value of η depends on the environment and would vary as:
Free space=−2
Urban=−2.7 to 3.5
With obstructions=−4 to 6

As such, an application 320 on the user device 300 may allow the user 400 to manually toggle between observed free space, urban area, with obstructions. In another preferred embodiment, η may be defined by the master central device 300 using the device's 300 known communication standard.

A BLE connection interval 540 is the time between two data transfer events (BLE connection events) between the user device 300 and the orthodontic device 100. The value ranges from 7.5 ms to 4 secs (with increments of 1.25 ms). A BLE stack on the BLE SoC 200 may allow setting a minimum and maximum connection interval. The maximum connection interval value provided by the orthodontic device 100 allows the user device 300 to choose an accepted value within the range rather than choosing a value different than the minimum and possibly outside the acceptable range for the orthodontic device 100. Once a connection between the user device 300 and orally insertable orthodontic device 100 are no longer able to connect, the user device's 300 application 320 would record the event including timing and location data.

FIG. 4B models a geographic space 600 with multiple advertising devices 610, 620, 630, 100 spread unevenly across the space 600. “X” represents the user 400 and his path across the space 600. In the initial position inside the inner-most circle, the user device 300, registers four devices that are advertising 610, 620,630, 100. The advertising devices 500 transmit advertising data 505 including an alphanumeric string 520 and device address 530. The advertising 505 allows the central master device 300 to then measures an RSSI 510 between the user device 300 and the advertising devices 500, as discussed in FIG. 4A. In this example, the key data collected for the four advertising devices 500 is:
DMM 72134 −56 dBm (610)
Lisa's iPhone −62 dBm (620)
LiftMaster −64 dBm (630)
Retainer −92 dBm (100)

The underlined reference numerals in the draws show an advertising 500 device superimposed over the radial geographic surface.

The user device 300 may also marry the advertising data 505 with the GPS location of the central master device 300.

The advertising devices 500 may be advertising 505 across multiple standards such as Wi-Fi and Bluetooth. This data only provides the relative multiple distance measurements of the advertising devices 500 from the user device 300 in a concentric manner. The data can neither provide the location or the direction in which the user 400 should move to locate the orthodontic device 100. The data in this example does show that the orthodontic device 100 is located the furthest of all the advertising devices 500 from the user 400.

The application 320 may then instruct the user 400 to move, with the master central device 300 within the geographic space 600. In the present example, the user, marked ‘X’ moves from the centermost circle to two circles away, also marked with ‘X’. Using the movement direction of the user 400, the application 320 can mark a False North/South axis 650 on a virtual map. At least one axis 650 line is created by the user device's application 320 that is unassociated with true north—the application 320 use the axis 650 to determine relative distances for the advertising devices 500. Thus, this axis 650 provides the first step in determining the location of the orally insertable orthodontic device 100. As the user 400 moves to a new location, the distance value of the advertising devices 500, including the orthodontic device 100, provide signal strength 510 to the application 320. This is then mapped, using triangulation techniques around the axis 650. The new example data in the example show the signal strengths 510 to have changed to:
DMM 72134 −72 dBm (610)
Lisa's iPhone −42 dBm (620)
LiftMaster −87 dBm (630)
Retainer −99 dBm (100)

In this example the RSSI calculation 510 tell us that the user 400 is moving further away from the ‘Retainer’ 100, further from ‘DMM 72134’ 610, closer to ‘Lisa's iPhone’ 620, and further away from ‘LiftMaster’ 630.

Using the False North/South axis 650, the application 320 can determine the relative positions between the advertising devices 500 and the axis 650 by calculating the angular information between the axis and changes in the dBm 510 of the advertising devices 500. This generates a spatial relationship for all the devices 500, 300. The False North/South axis 650 generates a line of direction of the orthodontic device 100 for the application to report to the user. The signal strength 510 is used to calculate the distance. With a direction and distance, the application 320 is now able to direct the patient 400 to the location of the lost orthodontic device 100.

By using the master central device's 300 accelerometers, gyroscopes, and compasses, the application 320 would be capable of reporting a countdown of the number walking steps to the orthodontic device 100.

Although the present disclosure and its advantages have been described in detail, it should be understood that various changes, substitutions and alterations can be made herein without departing from the spirit and scope of the disclosure as defined by the appended claims. Moreover, the scope of the present application is not intended to be limited to the particular embodiments of the process, machine, manufacture, composition of matter, means, methods and steps described in the specification. As one of ordinary skill in the art will readily appreciate from the disclosure, processes, machines, manufacture, compositions of matter, means, methods, or steps, presently existing or later to be developed that perform substantially the same function or achieve substantially the same result as the corresponding embodiments described herein may be utilized according to the present disclosure. Accordingly, the appended claims are intended to include within their scope such processes, machines, manufacture, compositions of matter, means, methods, or steps.

In the foregoing description, and the following claims, method steps and/or actions are described in a particular order for the purposes of illustration. It should be appreciated that in alternate embodiments, the method steps and/or actions may be performed in a different order than that described. Additionally, the methods described above may be embodied in machine-executable instructions stored on one or more machine-readable mediums, such as disk drives, thumb drives or CD-ROMs. The instructions may be used to cause the machine (e.g., computer processor) programmed with the instructions to perform the method. Alternatively, the methods may be performed by a combination of hardware and software. While illustrative and presently preferred embodiments of the invention have been described in detail herein, it is to be understood that the inventive concepts may be otherwise variously embodied and employed, and that the appended claims are intended to be construed to include such variations, except as limited by the prior art.

Benefits, other advantages, and solutions to problems have been described herein with regard to specific embodiments. However, the advantages, associated benefits, specific solutions to problems, and any element(s) that may cause any benefit, advantage, or solution to occur or become more pronounced are not to be construed as critical, required, or essential features or elements of any or all the claims of the invention. As used herein, the terms “comprises”, “comprising”, or any other variation thereof, are intended to cover a non-exclusive inclusion, such that a process, method, article, or apparatus composed of a list of elements that may include other elements not expressly listed or inherent to such process, method, article, or apparatus.

### ADVANTAGES

From the description, above, a number of advantages become evident for the “Orally Insertable Device with Usage and Location Tracking.” The present invention provides all new benefits for systems and business methods, including:
a) reducing the power requirements for the orthodontic device;
b) providing a False North/South axis to locate an orally insertable orthodontic device;
c) issuing a communication when the device is placed in the mouth;
d) issuing a communication when the orthodontic device is removed from the mouth;
e) issuing advertising data;
f) providing an automated diary of usage by the patient;
g) issuing electronic reminders to the patient that the orally insertable orthodontic device has not been replaced after an event;
h) provides locating solutions to locate a retainer within a geographic space;
i) provide mapping solutions to show the location of an orally insertable orthodontic device in a large geographic space;
j) generates power while the orally insertable orthodontic device is in the mouth.

What is claimed is:

1. An orally insertable orthodontic device with the device components comprising:
a. a power generator;
b. a battery;
c. the power generator with one or more connectors to the battery;
d. the power generator comprises titanium
e. a deforming switch comprising two or more deforming contacts;
f. the battery connected to the deforming switch and a Bluetooth System on Chip;
g. The Bluetooth System on Chip transmitting switch transmitting data;
whereby, the deforming switch allows the user device to record when the device is removed and inserted in the mouth and;
whereby, the deforming switch allows the user device to track the location of the orally insertable orthodontic device.

2. A method of claim 1, wherein the connector is wireless.

3. A method of claim 1, wherein the connector comprises a converter.

4. A method of claim 1, wherein the connector comprises an inverter.

5. A method of claim 1, wherein the connector comprises a rectifier.

6. A method of claim 1, wherein the Bluetooth System on Chip comprises a radio.

7. A method of claim 1, wherein the Bluetooth System on Chip comprises one or more peripherals.

8. A method of claim 1, wherein the Bluetooth System on Chip comprises memory.

9. A method of claim 1, wherein the Bluetooth System on Chip comprises a processor.

10. A method of claim 1, wherein the Bluetooth System on Chip comprises one or more radios.
